SEQUENCE LISTING (14 sequences)

SEQ ID NO: 1 - DNA, 24 bp, Artificial Sequence
  Synthetic DNA
agctgttgcagccctggcggtaag

SEQ ID NO: 2 - DNA, 24 bp, Artificial Sequence
  Synthetic DNA
aacaataaaaaagggcagaaaata

SEQ ID NO: 3 - DNA, 32 bp, Artificial Sequence
  Synthetic DNA
tgcccwaccggyatygcncacaccttcatggc

SEQ ID NO: 4 - DNA, 23 bp, Artificial Sequence
  Synthetic DNA
gcngcgaasggratngcrccytc

SEQ ID NO: 5 - DNA, 16 bp, Artificial Sequence
  Synthetic DNA
gtaaaacgacggccag

SEQ ID NO: 6 - DNA, 17 bp, Artificial Sequence
  Synthetic DNA
caggaaacagctatgac

SEQ ID NO: 7 - DNA, 30 bp, Artificial Sequence
  Synthetic DNA
gctaccctgctgcgcaagaagctgttcacc

SEQ ID NO: 8 - DNA, 32 bp, Artificial Sequence
  Synthetic DNA
agagcaagaaaacggcaagtcttcctggctgc

SEQ ID NO: 9 - DNA, 30 bp, Artificial Sequence
  Synthetic DNA
tcatcgcggccttccgcgttttgcgtcagg

SEQ ID NO: 10 - DNA, 30 bp, Artificial Sequence
  Synthetic DNA
atccgcagccatgaaggtgtgagcgatacc

SEQ ID NO: 11 - DNA, 35 bp, Artificial Sequence
  Synthetic DNA
gtacatattgtcgttagaacgcgtaatacgactca

SEQ ID NO: 12 - DNA, 35 bp, Artificial Sequence
  Synthetic DNA
cgttagaacgcgtaatacgactcactatagggaga

SEQ ID NO: 13 - DNA, 3378 bp, Brevibacterium lactofermentum
  CDS (881)..(2944)
gtggtaaaggcatcaatgtcgcccacgctgtcttgcttgcgggctttgaaaccttggctgtgttcccagccggcaagctcgaccccttcgtcccactggtccgcgacatcggcttgcccgtggaaactgttgtgatcaacaacaacgtccgcaccaacaccacagtcaccgaaccggacggcaccaccaccaagctcaacggccccggcgcaccgctcagcgagcagaagctccgtagcttggaaaaggtgcttatcgacgcgctccgccccgaagtcacctgggttgtcttggcgggctcgctgccaccaggggcaccagttgactggtacgcgcgtctcaccgcgttgatccattcagcacgccctgacgttcgcgtggctgtcgatacctccgacaagccactgatggcgttgggcgagagcttggatacacctggcgctgctccgaacctgattaagccaaatggtctggaactgggccagctggctaacactgatggtgaagagctggaggcgcgtgctgcgcaaggcgattacgacgccatcatcgcagctgcggacgtactggttaaccgtggcatcgaacaggtgcttgtcaccttgggtgccgctggagcggtgttggtcaacgcagaaggtgcgtggactgctacttctccaaagattgatgttgtatccaccgttggagctggagacagtgctcttgcaggttttgttatcgcacgttcccagaagaaaacactggaggaatctctgctgaatgccgtgtcttacggctcgactgcggcgtctcttcctggcactaccattcctcgtcctgaccaactcgccacaactggtgcaacggtcacccaagtcaaaggattgaaagaatcagcatgaatagcgtaattaattcctcgcttgtccggctggatgtcgatttcggcgactccaccacggatgtcatcaacaaccttgccactgttattttcgacgctggccgagcttcctccgccgacgcccttgccaaagacgcgctggatcgtgaagcaaagtccggcaccggtgtccccggtcaagttgctatcccccactgccgttccgaagccgtatctgtccctaccttgggctttgctcgcctgagcaagggtgtggacttcagcggacctgacggcgatgccaacttggtgttcctcattgcagcacctgctggcggcggcaaagagcacctgaagatcctgtccaaactcgctcgctccttggtgaagaaggatttcatcaaggctctgcaggaagccaccaccgagcaggaaatcgtcgacgttgtcgatgccgtgctcaacccagcaccaaaaaccaccgagccagctgcagctccggctgcgacggcggttgctgagagtggggcggcgtcgacaagcgttactcgtatcgtggcaatcaccgcatgcccaaccggtatcgcacacacctacatggctgcggattccctgacgcaaaacgcggaaggccgcgatgatgtggaactcgttgtggagactcagggctcttccgctgtcaccccagttgatccgaagatcatcgaagctgccgacgccgtcatcttcgccaccgacgtgggagttaaagaccgcgagcgtttcgctggcaagccagtcattgaatccggcgtcaagcgcgcgatcaatgagccagccaagatgatcgacgaggccatcgcagcctccaagaacccaaacgcccgcaaggtttccggttccggtgtcgcggcatctgctgaaaccaccggcgagaagctcggctggggcaagcgcatccagcaggcagtcatgaccggcgtgtcctacatggttccattcgtagctgccggcggcctcctgttggctctcggcttcgcattcggtggatacgacatggcgaacggctggcaagcaatcgccacccagttctccctgaccaacctgccaggcaacaccgtcgatgttgacggcgtggccatgaccttcgagcgttcaggcttcctgctgtacttcggcgcagtcctgttcgctaccggccaagcagccatgggcttcatcgtggcagcactgtctggctacaccgcatacgcacttgctggacgccctggcatcgcgccgggcttcgtcggtggcgccatctccgtcaccatcggcgctggcttcattggtggtctggttaccggtatcttggctggtctcattgccctgtggattggctcctggaaggtgccacgcgtggtgcagtcactgatgcctgtggtcatcatcccgctacttacctcagtggttgttggactcgtcatgtacctcctgctgggtcgcccactcgcatccatcatgactggtttgcaggactggctatcgtcaatgtccggaagctccgccatcttgctgggtatcatcttgggcctcatgatgtgtttcgacctcggcggaccagtaaacaaggcagcctacctctttggtaccgcaggcctgtctaccggcgaccaagcttccatggaaatcatggccgcgatcatggcagctggcatggtcccaccaatcgcgttgtccattgctaccctgctgcgcaagaagctgttcaccccagcagagcaagaaaacggcaagtcttcctggctgcttggcctggcattcgtctccgaaggtgccatcccattcgccgcagctgacccattccgtgtgatcccagcaatgatggctggcggtgcaaccactggtgcaatttccatggcactgggcgtcggctctcgggctccacacggcggtatcttcgtggtctgggcaatcgaaccatggtggggctggctcatcgcacttgcagcaggcaccatcgtgtccaccatcgttgtcatcgcactgaagcagttctggccaaacaaggccgtcgctgcagaagtcgcgaagcaagaagcagctgcggccgccgtagccgcataaccctgatgtctggtcggacattgtttttgcttccggtaacgtggcaaaacgaacaatgtctcactagactaaagtgagatccacattaaatcccctccgttgggggtttaactaacaaatcgctgcgccctaatccgttcggatgaacggcgtagcaacacgaaaggacactttccatggcttccaagactgtaaccgtcggttcctccgttggcctgcacgcacgtccagcatccatcatcgctgaagcggctgctgagtacgacgacgaaatcttgctgaccctggttggctccgatgatgacgaagagaccgacgcttcctcttccctcatgatcatggcgctgggtgcagagcacggcaacgaagtaaccgtcacctccgacaacgctgaagctgttgagaagatcgctgcgcttatcgcacaggac

SEQ ID NO: 14 - protein, 688 aa, Brevibacterium lactofermentum
MNSVINSSLVRLDVDFGDSTTDVINNLATVIFDAGRASSADALAKDALDREAKSGTGVPGQVAIPHCRSEAVSVPTLGFARLSKGVDFSGPDGDANLVFLIAAPAGGGKEHLKILSKLARSLVKKDFIKALQEATTEQEIVDVVDAVLNPAPKTTEPAAAPAATAVAESGAASTSVTRIVAITACPTGIAHTYMAADSLTQNAEGRDDVELVVETQGSSAVTPVDPKIIEAADAVIFATDVGVKDRERFAGKPVIESGVKRAINEPAKMIDEAIAASKNPNARKVSGSGVAASAETTGEKLGWGKRIQQAVMTGVSYMVPFVAAGGLLLALGFAFGGYDMANGWQAIATQFSLTNLPGNTVDVDGVAMTFERSGFLLYFGAVLFATGQAAMGFIVAALSGYTAYALAGRPGIAPGFVGGAISVTIGAGFIGGLVTGILAGLIALWIGSWKVPRVVQSLMPVVIIPLLTSVVVGLVMYLLLGRPLASIMTGLQDWLSSMSGSSAILLGIILGLMMCFDLGGPVNKAAYLFGTAGLSTGDQASMEIMAAIMAAGMVPPIALSIATLLRKKLFTPAEQENGKSSWLLGLAFVSEGAIPFAAADPFRVIPAMMAGGATTGAISMALGVGSRAPHGGIFVVWAIEPWWGWLIALAAGTIVSTIVVIALKQFWPNKAVAAEVAKQEAAAAAVAA

(19) United States
(12) Patent Application Publication
(10) Pub. No.: US 2004/0121428 A1
(43) Pub. Date: Jun. 24, 2004

(54) Process for producing l-amino acid and novel gene

(76) Inventors: Masakazu Sugimoto, Kawasaki-shi (JP); Yuta Nakai, Kawasaki-shi (JP); Hisao Ito, Kawasaki-shi (JP); Osamu Kurahashi, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/148,898

(22) PCT Filed: Dec. 22, 2000
(86) PCT No.: PCT/JP00/09164

(30) Foreign Application Priority Data
Dec. 24, 1999 (JP) 11368096

(51) Int. Cl. C12P 21/06

(52) U.S. Cl. 435/69.1

(57) ABSTRACT

A gene coding for fructose phosphotransferase is introduced into a coryneform bacterium having an ability to produce an L-amino acid such as L-lysine or L-glutamic acid to enhance fructose phosphotransferase activity and thereby improve the L-amino acid producing ability.

### TECHNICAL FIELD

The present invention relates to methods for producing L-amino acids by fermentation, in particular, methods for producing L-lysine and L-glutamic acid, as well as microorganisms and a novel gene used for the methods. There are widely used L-lysine as additive for animal feed and so forth, and L-glutamic acid as raw materials of seasonings and so forth.

### BACKGROUND ART

L-Amino acids such as L-lysine and L-glutamic acid are industrially produced by fermentation by using coryneform bacteria that belong to the genus Brevibacterium, Corynebacterium or the like and have abilities to produce these L-amino acids. In order to improve the productivity of these coryneform bacteria, strains isolated from nature or artificial mutants of such strains have been used.

Further, various techniques have been disclosed for increasing the L-amino acid producing abilities by using recombinant DNA techniques to enhance L-amino acid biosynthetic enzymes. For example, as for coryneform bacteria having L-lysine producing ability, it is known that the L-lysine producing ability of the bacteria can be improved by introduction of a gene coding for aspartokinase of which feedback inhibition by L-lysine and L-threonine is desensitised (mutant type lysC), dihydrodipicolinate reductase gene (dapB), dihydrodipicolinate synthase gene (dapA), diaminopimelate decarboxylase gene (lysA) and diaminopimelate dehydrogenase gene (ddh) (WO96/40934), lysA and ddh (Japanese Patent Laid-open Publication No. (Kokai) No. 9-322774), lysC, lysA and phosphoenolpyruvate carboxylase gene (ppc) (Japanese Patent Laid-open Publication No. No. 10-165180), mutant type lysC, dapB, dapA, lysA and aspartate aminotransferase gene (aspC) (Japanese Patent Laid-open Publication No. 10-215883).

Further, as for Escherichia bacteria, it is known that the L-lysine producing ability is improved by successively enhancing dapA, mutant type lysC, dapB and diaminopimelate dehydrogenase gene (ddh) (or tetrahydrodipicolinate succinylase gene (dapD) and succinyl diaminopimelate deacylase gene (dapE)) (WO 95/16042). Incidentally, in WO95/16042, tetrahydrodipicolinate succinylase is erroneously described as succinyl diaminopimelate transaminase.

Furthermore, it was reported that introduction of a gene coding for citrate synthase derived from Escherichia coli or Corynebacterium glutamicum was effective for enhancement of L-glutamic acid producing ability in Corynebacterium or Brevibacterium bacteria (Japanese Patent Publication (Kokoku) No. 7-121228). In addition, Japanese Patent Laid-open Publication No. 61-268185 discloses a cell harboring recombinant DNA containing a glutamate dehydrogenase gene derived from Corynebacterium bacteria. Furthermore, Japanese Patent Laid-open Publication No. 63-214189 discloses a technique for increasing L-glutamic acid producing ability by amplifying glutamate dehydrogenase gene, isocitrate dehydrogenase gene, aconitate hydratase gene and citrate synthase gene.

However, structure of a gene coding for fructose phosphotransferase has not been reported for coryneform bacteria, and utilization of a gene coding for fructose phosphotransferase for breeding of coryneform bacteria is also unknown so far.

In addition, a gene coding for fructose phosphotransferase of coryneform bacteria such as Brevibacterium bacteria has not been known.

### DISCLOSURE OF THE INVENTION

An object of the present invention is to provide a method for producing an L-amino acid such as L-lysine or L-glutamic acid by fermentation, which is further improved compared with conventional techniques, and a bacterial strain used for such a method. Further, another object of the present invention is to provide a gene coding for fructose phosphotransferase of coryneform bacteria, which can be suitably used for construction of such a strain as mentioned above.

The inventors of the present invention assiduously studies in order to achieve the aforementioned objects. As a result, they found that, if a gene coding for fructose phosphotransferase was introduced into a coryneform bacterium to amplify the fructose phosphotransferase activity, production amount of L-lysine or L-glutamic acid could be increased. Further, they also succeeded in isolating a gene coding for fructose phosphotransferase of Brevibacterium lactofermentum. Thus, they accomplished the present invention.

That is, the present invention provides the followings.

(1) A coryneform bacterium having enhanced intracellular fructose phosphotransferase activity and an ability to produce an L-amino acid.

(2) The coryneform bacteria according to (1), wherein the L-amino acid is selected from L-lysine, L-glutamic acid, L-threonine, L-isoleucine and L-serine.

(3) The coryneform bacterium according to (1), wherein the fructose phosphotransferase activity is enhanced by increasing copy number of a gene coding for fructose phosphotransferase in a cell of the bacterium.

(4) The coryneform bacterium according to (3), wherein the gene coding for fructose phosphotransferase is derived from an Escherichia bacterium.

(5) The coryneform bacteria according to (3), wherein the gene coding for fructose phosphotransferase is derived from a coryneform bacterium.

(6) A method for producing an L-amino acid, comprising the steps of culturing the coryneform bacterium according to any one of (1) to (5) in a medium to produce and accumulate the L-amino acid in culture and collecting the L-amino acid from the culture.

(7) The method according to (6), wherein the L-amino acid is selected from L-lysine, L-glutamic acid, L-threonine, L-isoleucine and L-serine.

(8) The method according to (6) or (7), wherein the medium contains fructose as a carbon source.

(9) A DNA coding for a protein defined in the following (A) or (B):

(A) a protein that has the amino acid sequence of SEQ ID NO: 14 in Sequence Listing,

(B) a protein that has the amino acid sequence of SEQ ID NO: 14 in Sequence Listing including substitution, deletion, insertion, addition or inversion of one or several amino acid residues and has fructose phosphotransferase activity.

(10) The DNA according to (9), which is a DNA defined in the following (a) or (b):

(a) a DNA containing the nucleotide sequence of the nucleotide numbers 881-2944 in the nucleotide sequence of SEQ ID NO: 13 in Sequence Listing,

(b) a DNA that hybridizes with the nucleotide sequence of the nucleotide numbers 881-2944 in the nucleotide sequence of SEQ ID NO: 13 in Sequence Listing or a probe that can be prepared from the nucleotide sequence under the stringent conditions, and codes for a protein having fructose phosphotransferase activity.

Hereafter, the present invention will be explained in detail.

<1> Coryneform Bacterium of the Present Invention

The coryneform bacterium of the present invention is a coryneform bacterium having an L-amino acid producing ability and enhanced intracellular fructose phosphotransferase activity. The L-amino acid may be L-lysine, L-glutamic acid, L-threonine, L-isoleucine, L-serine or the like. Among these, L-lysine and L-glutamic acid are preferred. Although embodiments of the present invention will be explained hereafter mainly for coryneform bacteria having L-lysine producing ability or L-glutamic acid producing ability, the present invention can be similarly used for any L-amino acid so long as the proper biosynthesis system of the desired L-amino acid locates downstream from fructose phosphotransferase.

The coryneform bacteria referred to in the present invention include the group of microorganisms defined in Bergey's Manual of Determinative Bacteriology, 8th edition, p.599 (1974), which are aerobic, gram-positive and non-acid-fast bacilli not showing sporogenesis ability. They include those having hitherto been classified into the genus Brevibacterium, but united into the genus Corynebacterium at present (Int. J. Syst. Bacteriol., 41, 255 (1981)), and also include bacteria belonging to the genus Brevibacterium or Microbacterium closely relative to the genus Corynebacterium. Examples of coryneform bacterium strain suitably used for the production of L-lysine or L-glutamic acid include, for example, the followings.

Corynebacterium acetoacidophilum ATCC 13870

Corynebacterium acetoglutamicum ATCC 15806

Corynebacterium callunae ATCC 15991

Corynebacterium glutamicum ATCC 13032

(Brevibacterium divaricatum) ATCC 14020

(Brevibacterium lactofermentum) ATCC 13869

(Corynebacterium lilium) ATCC 15990

(Brevibacterium flavum) ATCC 14067

Corynebacterium melassecola ATCC 17965

Brevibacterium saccharolyticum ATCC 14066

Brevibacterium immariophilum ATCC 14068

Brevibacterium roseum ATCC 13825

Brevibacterium thiogenitalis ATCC 19240

Microbacterium ammoniaphilum ATCC 15354

Corynebacterium thermoaminogenes AJ12340 (FERM BP-1539)

To obtain these strains, one can be provided them from, for example, the American Type Culture Collection (Address: 12301 Parklawn Drive, Rockville, Md. 20852, United States of America). That is, each strain is assigned its registration number, and one can request provision of each strain by utilizing its registration number. The registration numbers corresponding to the strains are indicated on the catalog of the American Type Culture Collection. Further, the AJ12340 strain was deposited at the National Institute of Bioscience and Human-Technology, Agency of Industrial Science and Technology, Ministry of International Trade and Industry (1-3 Higashi 1-Chome, Tsukuba-shi, Ibaraki-ken, Japan, postal code: 305-8566)) as an international deposit under the provisions of the Budapest Treaty.

Besides the aforementioned strains, mutant strains derived from these bacterial strains and having an ability to produce an L-amino acid such as L-lysine or L-glutamic acid can also be used for the present invention. Examples of such artificial mutant strains include mutant strains resistant to S-(2-aminoethyl)-cysteine (abbreviated as “AEC” hereinafter) (e.g., Brevibacterium lactofermentum AJ11082 (NRRL B-11470), refer to Japanese Patent Publication (Kokoku) Nos. 56-1914, 56-1915, 57-14157, 57-14158, 57-30474, 58-10075, 59-4993, 61-35840, 62-24074, 62-36673, 5-11958, 7-112437 and 7-112438), mutant strains requiring amino acids such as L-homoserine for their growth (Japanese Patent Publication Nos. 48-28078 and 56-6499), mutant strains resistant to AEC and further requiring amino acids such as L-leucine, L-homoserine, L-proline, L-serine, L-arginine, L-alanine and L-valine (U.S. Pat. Nos. 3,708,395 and 3,825,472), L-lysine producing mutant strains resistant to DL-α-amino-ε-caprolactam, α-amino-lauryllactam, aspartic acid analogue, sulfa drug, quinoid and N-lauroylleucine, L-lysine producing mutant strains resistant to oxaloacetate decarboxylase or a respiratory tract enzyme inhibitor (Japanese Patent Laid-open Publication Nos. 50-53588,.50-31093, 52-102498, 53-9394, 53-86089, 55-9783, 55-9759, 56-32995, 56-39778, Japanese Patent Publication Nos. 53-43591 and 53-1833), L-lysine producing mutant strains requiring inositol or acetatic acid (Japanese Patent Laid-open Publication Nos. 55-9784 and 56-8692), L-lysine producing mutant strains that are susceptible to fluoropyruvic acid or a temperature of 34° C. or higher (Japanese Patent Laid-open Publication Nos. 55-9783 and 53-86090), L-lysine producing mutant strains of Brevibacterium or Corynebacterium bacteria resistant to ethylene glycol (U.S. Pat. No. 4,411,997) and so forth.

Further, there can also be mentioned Corynebacterium acetoacidophilum AJ12318 (FERM BP-1172) (refer to U.S. Pat. No. 5,188,949) and so forth as coryneform bacteria having L-threonine producing ability, and Brevibacterium flavum AJ12149 (FERM BP-759) (refer to U.S. Pat. No. 4,656,135) and so forth as coryneform bacteria having L-isoleucine producing ability.

<2> Amplification of Fructose Phosphotransferase Activity

In order to amplify fructose phosphotransferase activity in a cell of coryneform bacterium, a recombinant DNA can be prepared by ligating a gene fragment coding for fructose phosphotransferase with a vector functioning in the bacterium, preferably a multi-copy vector, and introduced into a coryneform bacterium having an ability to produce L-lysine or L-glutamic acid to transform it. The copy number of the gene coding for fructose phosphotransferase in the cell of the transformant strain is thereby increased, and as a result, the fructose phosphotransferase activity is amplified. In Escherichia coli, fructose phosphotransferase is encoded by fruA gene.

Although the fructose phosphotransferase gene is preferably a gene derived from a coryneform bacterium, any of such genes derived from other organisms such as Escherichia bacteria can also be used.

The nucleotide sequence of fruA gene of Escherichia coli was already elucidated (Genbank/EMBL/DDBJ accession No. M23196), and therefore the fruA gene can be obtained by PCR (polymerase chain reaction, refer to White, T. J. et al., Trends Genet.5, 185 (1989)) using primers prepared based on the nucleotide sequence, for example, the primers shown in Sequence Listing as SEQ ID NOS: 1 and 2 and chromosomal DNA of Escherichia coli as a template.

Further, the fruA gene derived from a coryneform bacterium such as Brevibacterium lactofermentum can also be obtained as a partial sequence by selecting a region showing high homology among amino acid sequences expected from known fruA genes such as those of Bacillus subtilis, Escherichia coli, Mycoplasma genitalium and Xanthomonas compestris, preparing primers for PCR based on the amino acid sequence of that region and performing PCR using Brevibacterium lactofermentum as a template. As examples of the aforementioned primers, the oligonucleotides shown as SEQ ID NO: 3 and SEQ ID NO: 4 can be mentioned.

Then, by utilizing the partial sequence of the fruA gene obtained as described above, the 5′ unknown region and 3′ unknown region of the fruA gene are obtained by means of inverse PCR (Genetics, 120, 621-623 (1988)), a method using LA-PCR In Vitro Cloning Kit (Takara Shuzo) or the like. When LA-PCR In Vitro Cloning Kit is used, the 3′ unknown region of fruA gene can be obtained by, for example, performing PCR using the primers shown as SEQ ID NOS: 5 and 9 as primary PCR and PCR using the primers shown as SEQ ID NOS: 6 and 10 as secondary PCR. Further, the 5′ unknown region of fruA gene can be obtained by, for example, performing PCR using the primers shown as SEQ ID NOS: 7 and 9 as primary PCR and PCR using the primers shown as SEQ ID NOS: 8 and 10 as secondary PCR. The nucleotide sequence of the DNA fragment including the full length of fruA gene obtained as described above is shown as SEQ ID NO: 13. Further, the amino acid sequence translated from an open reading frame deduced from the above nucleotide sequence is shown as SEQ ID NO: 14.

Furthermore, since the fruA gene of Brevibacterium lactofermentum and the nucleotide sequences of the franking regions thereof are elucidated by the present invention, a DNA fragment containing the full length of the fruA gene can be obtained by PCR using oligonucleotides designed based on the nucleotide sequences of those flanking regions.

Genes coding for fructose phosphotransferase of other bacteria can also be obtained in a similar manner.

The fruA gene of the present invention may be one coding for fructose phosphotransferase including substitution, deletion, insertion, addition or inversion of one or several amino acids at one or more sites, so long as the fructose phosphotransferase activity of the encoded protein is not degraded. Although the number of “several” amino acids referred to herein differs depending on position or type of amino acid residues in the three-dimensional structure of the protein, it may be specifically 2 to 200, preferably 2 to 50, more preferably 2 to 20.

A DNA coding for the substantially same protein as the aforementioned fructose phosphotransferase can be obtained by, for example, modifying the nucleotide sequence of fruA by means of the site-directed mutagenesis method so that one or more amino acid residues at a specified site should involve substitution, deletion, insertion, addition or inversion. A DNA modified as described above may also be obtained by a conventionally known mutagenesis treatment. The mutagenesis treatment includes a method of treating a DNA before the mutagenesis treatment in vitro with hydroxylamine or the like, and a method for treating a microorganism such as an Escherichia bacterium harboring a DNA before the mutagenesis treatment by ultraviolet irradiation or with a mutagenizing agent used for a usual mutagenesis treatment such as N-methyl-N′-nitro-N-nitrosoguanidine (NTG) and nitrous acid.

A DNA coding for substantially the same protein as fructose phosphotransferase can be confirmed by expressing such a DNA having a mutation as described above in an appropriate cell, and investigating activity of the expressed product. A DNA coding for substantially the same protein as fructose phosphotransferase can also be obtained by isolating a DNA that is hybridizable with a probe having a nucleotide sequence comprising, for example, the nucleotide sequence corresponding to nucleotide numbers of 881 to 2944 of the nucleotide sequence shown in Sequence Listing as SEQ ID NO: 13 or a part thereof, under the stringent conditions, and codes for a protein having the fructose phosphotransferase activity from a DNA coding for fructose phosphotransferase having a mutation or from a cell harboring it. The “stringent conditions referred to herein are conditions under which so-called specific hybrid is formed, and non-specific hybrid is not formed. It is difficult to clearly express these conditions by using any numerical value. However, for example, the stringent conditions are exemplified by a condition under which DNAs having high homology, for example, DNAs having homology of not less than 50% are hybridized with each other, but DNAs having homology lower than the above are not hybridized with each other. Alternatively, the stringent conditions are exemplified by a condition under which DNAs are hybridized with each other at a salt concentration corresponding to an ordinary condition of washing in Southern hybridization, i.e., 1×SSC, 0.1% SDS, preferably 0.1×SSC, 0.1% SDS, at 60° C.

As the probe, a partial sequence of the nucleotide sequence of SEQ ID NO: 13 can also be used. Such a probe may be prepared by PCR using oligonucleotides produced based on the nucleotide sequence of SEQ ID NO: 13 as primers, and a DNA fragment containing the nucleotide sequence of SEQ ID NO: 13 as a template. When a DNA fragment in a length of about 300 bp is used as the probe, the conditions of washing for the hybridization consist of, for example, 50° C., 2×SSC and 0.1% SDS.

Genes that are hybridizable under such conditions as described above includes those having a stop codon in the genes, and those having no activity due to mutation of active center. However, such genes can be easily distinguished by ligating each gene with a commercially available activity expression vector, and measuring the fructose phosphotransferase activity by the method described in Mori, M. & Shiio, I., Agric. Biol. Chem., 51, 129-138 (1987).

Specific examples of the DNA coding for a protein substantially the same as fructose phosphotransferase include a DNA coding for a protein that has homology of preferably 55% or more, more preferably 60% or more, still more preferably 80% or more, with respect to the amino acid sequence shown as SEQ ID NO: 14 and has fructose phosphotransferase activity.

The chromosomal DNA can be prepared from a bacterium, which is a DNA donor, for example, by the method of Saito and Miura (refer to H. Saito and K. Miura, Biochem. Biophys. Acta, 72, 619 (1963); Text for Bioengineering Experiments, Edited by the Society for Bioscience and Bioengineering, Japan, pp.97-98, Baifukan, 1992) or the like.

If the gene coding for fructose phosphotransferase amplified by the PCR method is ligated to a vector DNA autonomously replicable in a cell of Escherichia coli and/or coryneform bacteria to prepare a recombinant DNA and this is introduced into Escherichia coli, subsequent procedures become easy. As the vector autonomously replicable in a cell of Escherichia coli, a plasmid vector, especially such a vector autonomously replicable in a cell of host is preferred, and examples of such a vector include pUC19, pUC18, pBR322, pHSG299, pHSG399, pHSG398, RSF1010 and so forth.

Examples of the vector autonomously replicable in a cell of coryneform bacteria include pAM330 (refer to Japanese Patent Laid-open Publication No. 58-67699), pHM1519 (refer to Japanese Patent Laid-open Publication No. 58-77895) and so forth. Moreover, if a DNA fragment having an ability to make a plasmid autonomously replicable in coryneform bacteria is taken out from these vectors and inserted into the aforementioned vectors for Escherichia coli, they can be used as a so-called shuttle vector autonomously replicable in both of Escherichia coli and coryneform bacteria. Examples of such a shuttle vector include those mentioned below. There are also indicated microorganisms that harbor each vector, and accession numbers thereof at the international depositories are shown in the parentheses, respectively.

pAJ655 Escherichia coli AJ11882 (FERM BP-136) Corynebacterium glutamicum SR8201 (ATCC 39135)

pAJ1844 Escherichia coli AJ11883 (FERM BP-137) Corynebacterium glutamicum SR8202 (ATCC 39136)

pAJ611 Escherichia coli AJ11884 (FERM BP-138)

pAJ3148 Corynebacterium glutamicum SR8203 (ATCC 39137)

pAJ440 Bacillus subtilis AJ11901 (FERM BP-140)

pHC4 Escherichia coli AJ12617 (FERM BP-3532)

In order to prepare a recombinant DNA by ligating a gene coding for fructose phosphotransferase and a vector that can function in a cell of coryneform bacterium, the vector is digested with a restriction enzyme corresponding to the terminus of the gene coding for fructose phosphotransferase. Ligation is usually performed by using a ligase such as T4 DNA ligase.

To introduce the recombinant DNA prepared as described above into a microorganism, any known transformation methods that have hitherto been reported can be employed. For instance, employable are a method of treating recipient cells with calcium chloride so as to increase the permeability of DNA, which has been reported for Escherichia coli K-12 (Mandel, M. and Higa, A., J. Mol. Biol., 53, 159 (1970)), and a method of preparing competent cells from cells which are at the growth phase followed by introducing the DNA thereinto, which has been reported for Bacillus subtilis (Duncan, C. H., Wilson, G. A. and Young, F. E., Gene, 1, 153 (1977)). In addition to these, also employable is a method of making DNA-recipient cells into protoplasts or spheroplasts, which can easily take up recombinant,DNA, followed by introducing the recombinant DNA into the cells, which is known to be applicable to Bacillus subtilis, actinomycetes and yeasts (Chang, S. and Choen, S. N., Molec. Gen. Genet., 168, 111 (1979); Bibb, M. J., Ward, J. M. and Hopwood, O. A., Nature, 274, 398 (1978); Hinnen, A., Hicks, J. B. and Fink, G. R., Proc. Natl. Sci., USA, 75, 1929 (1978)). The transformation method used in the examples mentioned in the present specification is the electric pulse method (refer to Japanese Patent Laid-open No. 2-207791).

Amplification of the fructose phosphotransferase activity can also be achieved by introducing multiple copies of a gene coding kor fructose phosphotransferase into chromosomal DNA of the host. In order to introduce multiple copies of the gene coding for fructose phosphotransferase into chromosomal DNA of a microorganism belonging to coryneform bacteria, homologous recombination is carried out by using a sequence whose multiple copies exist in the chromosomal DNA as targets. As sequences whose multiple copies exist in the chromosomal DNA, repetitive DNA or inverted repeats existing at the end of a transposable element can be used. Further, as disclosed in Japanese Patent Laid-open Publication No. 2-109985, it is also possible to incorporate the gene coding for fructose phosphotransferase into transposon, and allow it to be transferred to introduce multiple copies of the gene into the chromosomal DNA. According to any of these methods, the fructose phosphotransferase is amplified as a result of increase of copy number of the gene cording for fructose phosphotransferase in the transformant strain.

The amplification of fructose phosphotransferase activity can also be attained by, besides being based on the aforementioned gene amplification, replacing an expression regulatory sequence such as a promoter of the gene coding for fructose phosphotransferase on chromosomal DNA or plasmid with a stronger one (see Japanese Patent Laid-open Publication No. 1-215280). For example, lac promoter, trp promoter, trc promoter, tac promoter, P_{R} promoter and P_{L} promoter of lambda phage and so forth are known as strong promoters. Substitution of these promoters enhances expression of the gene coding for fructose phosphotransferase, and hence the fructose phosphotransferase activity is amplified.

In the coryneform bacterium of the present invention, in addition to the enhancement of fructose phosphotransferase activity, another enzyme involved in a biosynthetic pathway of another amino acid or the glycolysis system may also be enhanced by enhancing a gene for the enzyme. For example, examples of genes that can be used for production of L-lysine include a gene coding for the aspartokinase α-subunit protein or β-subunit protein of which synergistic feedback inhibition by L-lysine and L-threonine is desensitised (International Patent Publication WO94/25605), wild type phosphoenolpyruvate carboxylase gene derived from coryneform bacterium (Japanese Patent Laid-open Publication No. 60-87788), gene coding for wild type dihydrodipicolinate synthetase derived from coryneform bacterium (Japanese Patent Publication No. 6-55149) and so forth.

Further, examples of genes that can be used for production of L-glutamic acid include genes of glutamate dehydrogenase (GDH, Japanese Patent Laid-open Publication No. 61-268185), glutamine synthetase, glutamate synthase, isocitrate dehydrogenase (Japanese Patent Laid-open Publication Nos. 62-166890 and 63-214189), aconitate hydratase (Japanese Patent Laid-open Publication No. 62-294086), citrate synthase, pyruvate carboxylase (Japanese Patent Laid-open Publication Nos. 60-87788 and 62-55089), phosphoenolpyruvate carboxylase, phosphoenolpyruvate synthase, fructose phosphotransferase, phosphoglyceromutase, phosphoglycerate kinase, glyceraldehyde-3-phosphate dehydrogenase, triose phosphate isomerase, fructose bisphosphate aldolase, phosphofructokinase (Japanese Patent Laid-open Publication No. 63-102692), glucosephosphate isomerase and so forth.

Further, activity of an enzyme that catalyzes a reaction for producing a compound other than the desired L-amino acid by branching off from the biosynthetic pathway of the L-amino acid may be decreased or made deficient. For example, examples of an enzyme that catalyzes a reaction for producing a compound other than L-lysine by branching off from the biosynthetic pathway of L-lysine include homoserine dehydrogenase (refer to WO95/23864). Further, examples of an enzyme that catalyzes a reaction for producing a compound other than L-glutamic acid by branching off from the biosynthetic pathway of L-glutamic acid include α-ketoglutarate dehydrogenase, isocitrate lyase, phosphate acetyltransferase, acetate kinase, acetohydroxy acid synthase, acetolactate synthase, formate acetyltransferase, lactate dehydrogenase, glutamate decarboxylase, 1-pyrrolin dehydrogenase and so forth.

Furthermore, by imparting a temperature sensitive mutation for a biotin action suppressing substance such as surfactants to a coryneform bacterium having L-glutamic acid producing ability, L-glutamic acid can be produced in a medium containing an excessive amount of biotin in the absence of a biotin action suppressing substance (refer to WO96/06180). As an example of such a coryneform bacterium, the Brevibacterium lactofermentum AJ13029 strain disclosed in WO96/06180 can be mentioned. The AJ13029 strain was deposited at the Institute of Bioscience and Human-Technology, Agency of Industrial Science and Technology (1-3 Higashi 1-Chome, Tsukuba-shi, Ibaraki-ken, Japan, postal code: 305-8566) on Sep. 2, 1994, and given with an accession number of FERM P-14501, and then it was transferred to an international deposit under the provisions of the Budapest Treaty on Aug. 1, 1995, and given with an accession number of FERM BP-5189.

Furthermore, by imparting a temperature sensitive mutation for a biotin action suppressing substance such as surfactants to a coryneform bacterium having L-lysine and L-glutamic acid producing abilities, L-lysine and L-glutamic acid can be simultaneously produced in a medium containing an excessive amount of biotin in the absence of a biotin action suppressing substance (refer to WO96/06180). As an example of such a coryneform bacterium, the Brevibacterium lactofermentum AJ12933 strain disclosed in WO96/06180 can be mentioned. The AJ12933 strain was deposited at the Institute of Bioscience and Human-Technology, Agency of Industrial Science and Technology (1-3 Higashi 1-Chome, Tsukuba-shi, Ibaraki-ken, Japan, postal code: 305-8566) on Jun. 3, 1994, and given with an accession number of FERM P-14348, then it was transferred to an international deposit under the provisions of the Budapest Treaty on Aug. 1, 1995, and given with an accession number of FERM BP-5188.

<3> Production of L-Amino Acid

If a coryneform bacterium having amplified fructose phosphotransferase activity and an L-amino acid producing ability is cultured in a suitable medium, the L-amino acid is accumulated in the medium. For example, if a coryneform bacterium having amplified fructose phosphotransferase activity and L-lysine producing ability is cultured in a suitable medium, L-lysine is accumulated in the medium. Further, if a coryneform bacterium having amplified fructose phosphotransferase activity and L-glutamic acid producing ability is cultured in a suitable medium, L-glutamic acid is accumulated in the medium.

Furthermore, if a coryneform bacterium having amplified fructose phosphotransferase activity and L-lysine and L-glutamic acid producing abilities is cultured in a suitable medium, L-lysine and L-glutamic acid are accumulated in the medium. When L-lysine and L-glutamic acid are simultaneously produced by fermentation, an L-lysine producing bacterium may be cultured under an L-glutamic acid producing condition, or a coryneform bacterium having L-lysine producing ability and a coryneform bacterium having L-glutamic acid producing ability can be cultured as mixed culture (Japanese Patent Laid-open Publication No. No. 5-3793).

The medium used for producing L-amino acids such as L-lysine and L-glutamic acid by using the microorganism of the present invention is a usual medium that contains a carbon source, a nitrogen source, inorganic ions and other organic trace nutrients as required. As the carbon source, it is possible to use hydrocarbons such as glucose, lactose, galactose, fructose, sucrose, blackstrap molasses and starch hydrolysate; alcohols such as ethanol and inositol; or organic acids such as acetic acid, fumaric acid, citric acid and succinic acid. In the present invention, fructose is particularly preferred among these. Usually, in the production of L-amino acids by fermentation using coryneform bacteria, yield tends to be degraded if fructose is used as a carbon source of the medium. However, the microorganism used for the present invention efficiently produces an L-amino acid in a medium containing fructose as a carbon source. This effect is particularly remarkable in L-lysine production.

As the nitrogen source, there can be used inorganic or organic ammonium salts such as ammonium sulfate, ammonium nitrate, ammonium chloride, ammonium phosphate and ammonium acetate, ammonia, organic nitrogen such as peptone, meat extract, yeast extract, corn steep liquor and soybean hydrolysate, ammonia gas, aqueous ammonia and so forth.

As the inorganic ions (or sources thereof), added is a small amount of potassium phosphate, magnesium sulfate, iron ions, manganese ions and so forth. As for the organic trace nutrients, it is desirable to add required substances such as vitamin B₁, yeast extract and so forth in a suitable amount as required.

The culture is preferably performed under an aerobic condition attained by shaking, stirring for aeration or the like for 16 to 72 hours. The culture temperature is controlled to be at 30° C. to 45° C., and pH is controlled to be 5 to 9 during the culture. For such adjustment of pH, inorganic or organic acidic or alkaline substances, ammonia gas and so forth can be used.

Collection of L-amino acid from fermentation broth can be attained in the same manner as in usual production methods of L-amino acids. For example, collection of L-lysine can be usually performed by a combination of conventional techniques, for example, a method utilizing ion exchange resin, crystallization and others. Further, collection of L-glutamic acid can also be performed in a conventional manner, and it can be performed by, for example, a method utilizing ion exchange resin, crystallization or the like. Specifically, L-glutamic acid can be adsorbed on an anion exchange resin and isolated from it, or crystallized by neutralization. When both of L-lysine and L-glutamic acid are produced and used as a mixture, it is unnecessary to separate these amino acids from each other.

### BEST MODE FOR CARRYING OUT THE INVENTION

Hereafter, the present invention will be more specifically explained with reference to the following examples.

### EXAMPLE 1

Construction of Coryneform Bacterium Introduced with fruA Gene

<1> Cloning of fruA Gene of Escherichia coli JM109 Strain

The nucleotide sequence of the fruA gene of Escherichia coli had already been elucidated (Genbank/EMBL/DDBJ accession No. M23196). The primers shown in Sequence Listing as SEQ ID NOS: 1 and 2 were synthesized based on the reported nucleotide sequence, and the fructose phosphotransferase gene was amplified by PCR utilizing chromosome DNA of Escherichia coli JM109 strain as a template.

Among the synthesized primers, that of SEQ ID NO: 1 corresponded to the sequence of from the 1st to the 24th nucleotides of the nucleotide sequence of the fruA gene of Genbank/EMBL/DDBJ accession No. M23196, and that of SEQ ID NO: 2 corresponded to the sequence of from the 2000th to the 1977th nucleotides of the same.

The chromosome DNA of Escherichia coli JM109 strain was prepared by a conventional method (Text for Bioengineering Experiments, Edited by the Society for Bioscience and Bioengineering, Japan, pp.97-98, Baifukan, 1992). Further, for PCR, the standard reaction conditions described in “Forefront of PCR”, p.185 (compiled by Takeo Sekiya et al., Kyoritsu Shuppan, 1989).

The produced PCR product was purified in a conventional manner, then ligated to a plasmid pHC4 digested with SmaI by using a ligation kit (Takara Shuzo) and used for transformation of competent cells of Escherichia coli JM109 (Takara Shuzo). The cells were plated on L medium (10 g/L of Bacto trypton, 5 g/L of Bacto yeast extract, 5 g/L of NaCl, 15 g/L of agar, pH 7.2) containing 30 μg/ml of chloramphenicol and cultured overnight. Then, the emerged white colonies were picked up and separated into single colonies to obtain transformant strains. Plasmids were extracted from the obtained transformants, and a plasmid pHC4fru comprising the fruA gene ligated to the vector was obtained.

Escherichia coli harboring pHC4 was given with a private number of AJ12617 and deposited at the National Institute of Bioscience and Human-Technology, Agency of Industrial Science and Technology, Ministry of International Trade and Industry (1-3 Higashi 1-Chome, Tsukuba-shi, Ibaraki-ken, Japan, postal code: 305-8566) on Apr. 24, 1991 and given with an accession number of FERM P-12215. Then, it was transferred to an international deposit under the provisions of the Budapest Treaty based on Aug. 26, 1991 and given with an accession number of FERM BP-3532.

Then, in order confirm that the cloned DNA fragment coded for a protein having the fructose phosphotransferase activity, fructose phosphotransferase activity of the JM109 strain and the JM109 strain harboring pHC4fru was measured by the method described in Mori, M. & Shiio, I., Agric. Biol. Chem., 51, 129-138 (1987). As a result, it was confirmed that the JM109 strain harboring pHC4fru showed about 11 times higher fructose phosphotransferase activity compared with the JM109 strain not harboring pHC4fru, and thus it was confirmed that the fruA gene was expressed.

<2> Introduction of pHC4fru Into L-Glutamic Acid Producing Strain of Coryneform Bacterium and Production of L-Glutamic Acid

The Brevibacterium lactofermentum AJ13029 strain was transformed with the plasmid pHC4fru by the electric pulse method (refer to Japanese Patent Laid-open Publication No. 2-207791) to obtain a transformant strain. Culture for L-glutamic acid production was performed as follows by using the obtained transformant strain AJ13029/pHC4fru. Cells of the AJ13029/pHC4fru strain obtained after culture on CM2B plate medium containing 5 μg/ml of chloramphenicol were inoculated into an L-glutamic acid production medium having the following composition containing 5 μg/ml of chloramphenicol and cultured at 31.5° C. with shaking until the sugar in the medium was consumed. The obtained culture was inoculated into a medium having the same composition in 5% amount and cultured at 37° C. with shaking until the sugar in the medium was consumed. As a control, the Corynebacterium bacterium AJ13029 strain transformed with the previously obtained plasmid pHC4 autonomously replicable in Corynebacterium bacteria by the electric pulse method was cultured in the same manner as described above.

[L-Glutamic Acid Production Medium]

The following components are dissolved (in 1 L), adjusted to pH 8.0 with KOH and sterilized at 115° C. for 15 minutes.

| | | |
|---|---|---|
| Fructose | 150 | g |
| KH₂PO₄ | 2 | g |
| MgSO₄.7H₂O | 1.5 | g |
| FeSO₄.7H₂O | 15 | mg |
| MnSO₄.4H₂O | 15 | mg |
| Soybean protein hydrolyzed solution | 50 | mL |
| Biotin | 2 | mg |
| Thiamin hydrochloride | 3 | mg |

After completion of the culture, the amount of L-glutamic acid accumulated in the culture broth was measured with Biotech Analyzer AS-210 produced by Asahi Chemical Industry Co., Ltd. The results are shown in Table 1.

TABLE 1

| | Produced amount of L-glutamic acid |
|---|---|
| Strain | (g/L) |
| AJ13029/pHC4 | 18.5 |
| AJ13029/PHC4fru | 20.5 |

<3> Introduction of pHC4fru into L-Lysine Producing Strain of Coryneform Bacterium and Production of L-Lysine

The Brevibacterium lactofermentum AJ11082 strain was transformed with the plasmid pHC4fru by the electric pulse method (refer to Japanese Patent Laid-open Publication No. 2-207791) to obtain a transformant strain. Culture for L-lysine production was performed as follows by using the obtained transformant strain AJ11082/pHC4fru. Cells of the AJ11082/pHC4fru strain obtained after culture on CM2B plate medium containing 5 μg/ml of chloramphenicol were inoculated into an L-lysine production medium having the following composition containing 5 μg/ml of chloramphenicol and cultured at 31.5° C. with shaking until the sugar in the medium was consumed. As a control, the Corynebacterium bacterium AJ11082 strain transformed with the previously obtained plasmid pHC4 autonomously replicable in Corynebacterium bacteria by the electric pulse method was cultured in the same manner as described above.

The Brevibacterium lactofermentum AJ11082 was deposited at the Agricultural Research Service Culture Collection (1815 N. University Street, Peoria, Ill. 61604 U.S.A.) as an international deposit on Jan. 31, 1981 and given with an accession number of NRRL B-11470.

[L-Lysine Production Medium]

The following components are dissolved (in 1 L), adjusted to pH 8.0 with KOH, sterilized at 115° C. for 15 minutes, and then added with calcium carbonate separately subjected to dry sterilization.

| | | |
|---|---|---|
| Fructose | 100 | g |
| (NH₄)₂SO₄ | 55 | g |
| KH₂PO₄ | 1 | g |
| MgSO₄.7H₂O | 1 | g |
| Biotin | 500 | μg |
| Thiamine | 2000 | μg |
| FeSO₄.7H₂O | 0.01 | g |
| MnSO₄.4H₂O | 0.01 | g |
| Nicotinamide | 5 | mg |
| Protein hydrolysate (soybean milk) | 30 | mL |
| Calcium carbonate | 50 | g |

After completion of the culture, the amount of L-lysine accumulated in the culture broth was measured with Biotech Analyzer AS-210 produced by Asahi Chemical Industry Co., Ltd. The results are shown in Table 2.

TABLE 2

| Strain | Produced amount of L-lysine (g/L) |
|---|---|
| AJ11082/pHC4 | 24.9 |
| AJ11082/PHC4fru | 28.4 |

<4> Introduction of pHC4fru Into L-Lysine and L-Glutamic Acid Producing Strain of Coryneform Bacterium and Simultaneous Production of L-Lysine and L-Glutamic Acid

The Brevibacterium lactofermentum AJ12993 strain was transformed with the plasmid pHC4fru by the electric pulse method (refer to Japanese Patent Laid-open Publication No. 2-207791) to obtain a transformant strain. Culture for L-lysine and L-glutamic acid production was performed as follows by using the obtained transformant strain AJ12993/pHC4fru. Cells of the AJ12993/pHC4fru strain obtained after culture on CM2B plate medium containing 5 μg/ml of chloramphenicol were inoculated into the aforementioned L-lysine production medium containing 5 μg/ml of chloramphenicol and cultured at 31.5° C. After 12 hours from the start of the culture, the culture temperature was shifted to 34° C., and the culture was further continued with shaking until the sugar in the medium was consumed. As a control, the Corynebacterium bacterium AJ12993 strain transformed with the previously obtained plasmid pHC4 autonomously replicable in Corynebacterium bacteria by the electric pulse method was cultured in the same manner as described above.

After completion of the culture, the amounts of L-lysine and L-glutamic acid accumulated in the culture broth was measured with Biotech Analyzer AS-210 produced by Asahi Chemical Industry Co., Ltd. The results are shown in Table 3.

TABLE 3

| | | Produced amount of |
|---|---|---|
| | Produced amount of | L-glutamic acid |
| Strain | L-lysine (g/L) | (g/L) |
| AJ12993/pHC4 | 8.5 | 18.5 |
| AJ12993/PHC4fru | 9.7 | 20.3 |

### EXAMPLE 2

Isolation of fruA Gene of Brevibacterium lactofermentum

<1> Acquisition of fruA Gene Partial Fragment of Brevibacterium lactofermentum ATCC13869

A region showing high homology for amino acid sequence in FruA among those of Bacillus subtilis, Escherichia coli, Mycoplasma genitalium and Xanthomonas compestris was selected, a nucleotide sequence was deduced from the amino acid sequence of that region, and the oligonucleotides shown as SEQ ID NOS: 3 and 4 were synthesized. Separately, chromosomal DNA of the Brevibacterium lactofermentum ATCC13869 strain was prepared by using Bacterial Genome DNA Purification Kit (Advanced Genetic Technologies Corp.). Sterilized water was added to 0.5 μg of the chromosomal DNA, 20 pmol each of the oligonucleotides, 4 μl of DNTP mixture (DATP, dGTP, dCTP, dTTP, 2.5 mM each), 5 μl of 10×ExTaq Buffer (Takara Shuzo) and 1 U of ExTaq (Takara Shuzo) to prepare a PCR reaction mixture in a total volume of 50 μl. For this reaction mixture, PCR was performed for 25 cycles each consisting of denaturation at 98° C. for 10 seconds, annealing at 45° C. for 30 seconds and extension at 72° C. for 90 seconds by using Thermal Cycler TP 240 (Takara Shuzo), and the PCR product was subjected to agarose gel electrophoresis. As a result, it was found that the reaction mixture contained an about 1.2 kb band.

The reaction product was ligated to pCR2.1 (Invitrogen) by using Original TA Cloning Kit (Invitrogen). After the ligation, competent cells of Escherichia coli JM109 (Takara Shuzo) were transformed with the ligation mixture, then plated on L medium (10 g/L of Bacto Trypton, 5 g/L of Bacto Yeast Extract, 5 g/L of NaCl, 15 g/L of agar, pH 7.2) containing 10 μg/ml of IPTG (isopropyl-β-D-thiogalactopyranoside), 40 μg/ml of X-Gal (5-bromo-4-chloro-3-indolyl-β-D-galactoside) and 25 μg/ml of kanamycin, and cultured overnight. Then, the emerged white colonies were picked up and separated into single colonies to obtain transformant strains.

Plasmids were prepared from the obtained transformant strains by using the alkaline method (Text for Bioengineering Experiments, Edited by the Society for Bioscience and Bioengineering, Japan, p.105, Baifukan, 1992), and nucleotide sequences of the both ends of the inserted fragment were determined by the method of Sanger (J. Mol. Biol., 143, 161 (1980)) using the oligonucleotides shown as SEQ ID NOS: 5 and 6. Specifically, Big Dye Terminator Sequencing Kit (Applied Biosystems) was used for the nucleotide sequence determination, and analysis was performed by using Genetic Analyzer ABI 310 (Applied Biosystems). The determined nucleotide sequence was translated into an amino acid sequence, and it was compared with the amino acid sequences deduced from fruA genes of Bacillus subtilis, Escherichia coli, Mycoplasma genitalium and Xanthomonas compestris. As a result, it showed high homology, and thus the cloned fragment was determined to be the fruA gene derived from Brevibacterium lactofermentum.

<2> Determination of Whole Nucleotide Sequence of fruA Gene of Brevibacterium lactofermentum ATCC13869

The fragment contained in the plasmid prepared in the above <1> was a partial fragment of the fruA gene, and thus it was further necessary to determine the nucleotide sequence of the fruA gene in full length. While there were inverse PCR (Genetics, 120, 621-623 (1988), a method utilizing LA-PCR In Vitro Cloning Kit (Takara Shuzo) and so forth as methods for determining an unknown nucleotide sequence flanking to a known region, the unknown sequence was determined by using LA-PCR In Vitro Cloning Kit in this example. Specifically, the oligonucleotides shown as SEQ ID NOS: 7, 8, 9 and 10 were synthesized based on the nucleotide sequence determined in the above <1>, and the determination was performed according to the protocol of LA-PCR In Vitro Cloning Kit.

For the 3′ unknown region of the fruA gene partial fragment, chromosome DNA of the Brevibacterium lactofermentum ATCC13869 strain was treated with HindIII, ligated to HindIII Adapter contained in the kit and then used to perform PCR using the oligonucleotides of SEQ ID NOS: 7 and 11 as the primary PCR and PCR using the oligonucleotides of SEQ ID NOS: 8 and 12 as the secondary PCR. When this PCR product was subjected to agarose gel electrophoresis, a band of about 700 bp was observed. This band was purified by using Suprec ver. 2 (Takara Shuzo), and the nucleotide sequence of fruA gene contained in the 700 bp PCR product was determined by using the oligonucleotides of SEQ ID NOS: 8 and 12 in the same manner as described in <1>.

For the 5′ unknown region of the fruA gene partial fragment, chromosome DNA of the Brevibacterium lactofermentum ATCC13869 strain was treated with BamHI, ligated to Sau3AI Adapter contained in the kit and then used to perform PCR using the oligonucleotides of SEQ ID NOS: 9 and 11 as the primary PCR and PCR using the oligonucleotides of SEQ ID NOS: 10 and 12 as the secondary PCR. When this PCR product was subjected to agarose gel electrophoresis, a band of about 1500 bp was observed. This band was purified by using Suprec ver. 2 (Takara Shuzo), and the nucleotide sequence of fruA gene contained in the 1500 bp PCR product was determined by using the oligonucleotides of SEQ ID NOS: 10 and 12 in the same manner as described in <1>.

As for the nucleotide sequence determined as described above, the nucleotide sequence of about 3380 bp containing the fruA gene is shown in Sequence Listing as SEQ ID NO: 13. An amino acid sequence obtained by translating an open reading frame deduced from the above nucleotide sequence is shown as SEQ ID NO: 14. That is, a protein consisting of the amino acid sequence shown in Sequence Listing as SEQ ID NO: 14 is FruA of the Brevibacterium lactofermentum ATCC13869 strain. In addition, it is well known that a methionine residue at the N-terminus of a protein originates in ATG as a start codon and hence it does not relate to proper functions of the protein and removed by an action of peptidase after the translation in many cases. Removal of such a methionine residue might occur also in the aforementioned protein.

The above nucleotide sequence and amino acid sequence were compared with known sequences for homology. The used databases were GeneBank and SWISS-PROT. As a result, it was found that the DNA shown in Sequence Listing as SEQ ID NO: 13 was a novel gene in Corynebacterium bacteria showing homology with the already reported fruA genes.

The DNA shown as SEQ ID NO: 13 showed homology of 42.1%, 51.0%, 37.4% and 45.5% to fruA of Bacillus subtilis, Escherichia coli, Mycobacterium genetilium and Xanthomonas compestris, respectively, as the encoded amino acid. The nucleotide sequence and the amino acid sequence were analyzed by using Genetyx-Mac computer program (Software Development, Tokyo). The homology analysis was performed according to the method of Lipman and Peason (Science, 227, 1435-1441, 1985).

Industrial Applicability

According to the present invention, production ability of coryneform bacteria for L-amino acids such as L-lysine or L-glutamic acid can be improved. Further, according to the present invention, a novel fructose phosphotransferase gene derived from Brevibacterium lactofermentum is provided. This gene can be preferably used for breeding of coryneform bacteria suitable for production of L-amino acids.

What is claimed is:

1. A coryneform bacterium having enhanced intracellular fructose phosphotransferase activity and an ability to produce an L-amino acid.

2. The coryneform bacteria according to claim 1, wherein the L-amino acid is selected from L-lysine, L-glutamic acid, L-threonine, L-isoleucine and L-serine.

3. The coryneform bacterium according to claim 1, wherein the fructose phosphotransferase activity is enhanced by increasing copy number of a gene coding for fructose phosphotransferase in a cell of the bacterium.

4. The coryneform bacterium according to claim 3, wherein the gene coding for fructose phosphotransferase is derived from an Escherichia bacterium.

5. The coryneform bacteria according to claim 3, wherein the gene coding for fructose phosphotransferase is derived from a coryneform bacterium.

6. A method for producing an L-amino acid, comprising the steps of culturing the coryneform bacterium according to any one of claims 1 to 5 in a medium to produce and accumulate the L-amino acid in the culture and collecting the L-amino acid from the culture.

7. The method according to claim 6, wherein the L-amino acid is selected from L-lysine, L-glutamic acid, L-threonine, L-isoleucine and L-serine.

8. The method according to claim 6 or 7, wherein the medium contains fructose as a carbon source.

9. A DNA coding for a protein defined in the following (A) or (B):
(A) a protein that has the amino acid sequence of SEQ ID NO: 14 in Sequence Listing,
(B) a protein that has the amino acid sequence of SEQ ID NO: 14 in Sequence Listing including substitution, deletion, insertion, addition or inversion of one or several amino acid residues and has fructose phosphotransferase activity.

10. The DNA according to claim 9, which is a DNA defined in the following (a) or (b):
(a) a DNA containing the nucleotide sequence of the nucleotide numbers 881-2944 in the nucleotide sequence of SEQ ID NO: 13 in Sequence Listing,
(b) a DNA that hybridizes with the nucleotide sequence of the nucleotide numbers 881-2944 in the nucleotide sequence of SEQ ID NO: 13 in Sequence Listing or a probe that can be prepared from the nucleotide sequence under the stringent conditions, and codes for a protein having fructose phosphotransferase activity.
